(19) United States
(12) Patent Application Publication
(10) Pub. No.: US 2026/0228371 A1
(43) Pub. Date: Aug. 6, 2026

(54) CORONARY ARTERY BIFURCATION GEOMETRY MEASUREMENT SYSTEMS AND METHODS

(71) Applicant: THE BOARD OF TRUSTEES OF THE UNIVERSITY OF ARKANSAS, Little Rock, AR (US)

(72) Inventors: Morten JENSEN, Fayetteville, AR (US); Kaitlyn ELMER, Fayetteville, AR (US); Barry URETSKY, Fort Smith, AR (US); Adib CHAUS, Des Plaines, IL (US)

(21) Appl. No.: 19/156,817

(22) PCT Filed: Feb. 15, 2024
(86) PCT No.: PCT/US2024/015935

§ 371 Date: Aug. 14, 2025

Related U.S. Application Data
(60) Provisional application No. 63/445,940, filed on Feb. 15, 2023.

(51) Int. Cl. G06F 30/10; A61B 5/00; A61B 5/107; A61B 17/00; A61B 17/22; G06T 7/00; G06T 7/62

(52) U.S. Cl. CPC G06F 30/10; A61B 5/1075; A61B 5/7475; A61B 17/22; G06T 7/0012; G06T 7/62; A61B 2017/00526; A61B 2017/22001; G06T 2200/24; G06T 2207/30048; G06T 2207/30101; G06T 2207/30172

(57) ABSTRACT

Patient-specific angioplasty balloons based on novel geometric features of vessel bifurcation anatomy are extracted from medical images of the patient. The arterial bifurcation geometry extraction methods aid physicians and researchers with sizing for patient-specific features of vessel bifurcations. The calculated anatomic measurements of the coronary artery bifurcation include two measurements useful for designing patient-specific balloons: carina diameter and the tapering length.

### CROSS-REFERENCE TO RELATED APPLICATIONS

This application claims the benefit of U.S. Provisional Patent Application Ser. No. 63/445,940, filed on Feb. 15, 2023, and entitled “Coronary Artery Bifurcation Geometry Measurement Systems and Methods,” which is herein incorporated by reference in its entirety.

### BACKGROUND

Typically, coronary arteries with plaque build-up are treated using percutaneous coronary intervention (“PCI”) procedures such as balloon angioplasty procedures. Briefly, a balloon is expanded within the vessel to push the plaque outwards and thereby open the vessel. Stents are often used in conjunction with the balloon angioplasty to keep the vessel propped open following balloon removal.

PCI balloons are typically cylindrical in shape; however, at vessel bifurcations, traditional balloon and/or stent combinations might not match the local geometry. At the vessel bifurcation, the vessels tapers due to the main vessel splitting into two branches. Stents placed in the main vessel of the bifurcation may be placed far enough into the bifurcation core that they span the widest diameter of the bifurcation. Stents and balloon sizes should be chosen to accommodate this outward tapering. Stents have limits to individual cell expansion, and stent overexpansion past these limits distorts stent architecture. Underexpansion of the stent, on the other hand, is associated with adverse short and long-term clinical outcomes. As such, current cylindrical stents cannot easily accommodate to the bifurcation area. There is an unmet need, then, to develop balloons and stents that more accurately accommodate the bifurcation area.

### SUMMARY OF THE DISCLOSURE

The present disclosure addresses the aforementioned drawbacks by providing a method for generating anatomic measurements of a coronary artery bifurcation from medical image data. The method includes accessing medical image data with a computer system, where the medical image data depict a coronary artery bifurcation in a patient. The medical image data are then processed with the computer system to estimate a centerline of a main vessel of the coronary artery bifurcation. A carina point indicating a split between a main branch and a side branch of the coronary artery bifurcation is determined in the medical image data using the computer system, and a carina diameter in the medical image data is calculated using the main vessel center line and the carina point. A side branch ostium point indicating a split between the main vessel and the side branch of the coronary artery bifurcation is determined in the medical image data using the computer system, and a tapering length is calculated in the medical image data using the carina diameter and the side branch ostium point. The carina diameter and the tapering length are stored as patient-specific anatomic measurement data of the coronary artery bifurcation.

It is another aspect of the present disclosure to provide a method for designing a mold for use in manufacturing an angioplasty balloon. Medical image data of a patient are accessed using a computer system, where the medical image data depict a coronary artery bifurcation in a patient. The medical image data are processed with the computer system to: calculate a carina diameter in the medical image data by the computer system, where the carina diameter spans the coronary artery bifurcation from a first vessel wall to an opposed vessel wall passing through a carina point of the coronary artery bifurcation and perpendicular to a centerline of a main vessel of the coronary artery bifurcation; and calculate a tapering length in the medical image data by the computer system, where the tapering length is a distance between the carina diameter line and a parallel line in the medical image data intersecting a side branch ostium point. Balloon mold design data are then generated using the computer system, where the balloon model design data include instructions to manufacture a balloon mold including a conical portion having a large end, a small end, and a tapered portion extending between the large and the small end. The balloon mold design data are adjusted with the computer system by sizing the large end of the conical portion according to the carina diameter and sizing a distance between the large end and the small end of the conical portion based on the tapering length.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 illustrates example angiographic images of a coronary artery bifurcation.

FIG. 2 illustrates example main vessel, main branch, and side branch components of a coronary artery bifurcation.

FIG. 3 illustrates example anatomic measurements of a coronary artery bifurcation, including main vessel diameter, main branch diameter, side branch diameter, and various vessel angles.

FIG. 4 illustrates example anatomic features of a coronary artery bifurcation, including a carina point and side branch ostium point.

FIG. 5 illustrates an example model of a coronary artery bifurcation, showing anatomic measurements including vessel centerlines, side branch ostium and carina points, a carina diameter line and carina diameter, an ostium line and tapering length, among others.

FIG. 6 is a flowchart setting forth the steps of an example method for generating patient-specific anatomic measurement data of a coronary artery bifurcation from medical image data.

FIGS. 7A-7I show steps of generating various patient-specific anatomic measurement data of a coronary artery bifurcation from medical image data.

FIG. 8 illustrates a sectioned view of a customizable balloon mold (top) and mold assembled with one of two end pieces (bottom). Diameters A and C, length B, and angle α are all adjustable according to the required dimensions.

FIG. 9 illustrates a tapered stent delivery balloon designed based on patient-specific anatomic measurement data generated using the methods described in the present disclosure. (A) denotes the proximal main vessel diameter, (B) denotes the carina diameter (long axis), and (C) denotes the distal main vessel diameter (short axis).

FIG. 10 illustrates an example stent delivery balloon with a heart-shaped end designed according to the carina diameter measurement contained in patient-specific anatomic measurement data.

FIG. 11 illustrates an example of a heart-shaped stent delivery balloon.

FIG. 12 illustrates an example of a tapered stent delivery balloon.

FIG. 13 illustrates an example stent design that is designed based on patient-specific anatomic measurements of carina diameter (CD), proximal main vessel diameter (pMVD), and taper length (TL).

FIG. 14 illustrates an example of a tapered stent showing the long axis of the stent's distal end.

FIG. 15 is a flowchart setting forth the steps of an example method for generating disease-adjusted patient-specific anatomic measurement data of a coronary artery bifurcation from medical image data using a model.

FIG. 16 is a block diagram of an example system for generating patient-specific anatomic measurement data of a coronary artery bifurcation.

FIG. 17 is a block diagram of example components that can implement the system of FIG. 16.

### DETAILED DESCRIPTION

Described here systems and methods for designing patient-specific angioplasty balloons based on novel geometric features of vessel bifurcation anatomy extracted from medical images of the patient. The arterial bifurcation geometry extraction methods described in the present disclosure aid physicians and researchers with sizing for patient-specific features of vessel bifurcations. Additionally or alternatively, the disclosed systems and methods can be used to analyze and provide measurements of pre-manufactured angioplasty balloons. In this context, pre-manufactured angioplasty balloons can be matched to the particular anatomy of a patient, such that a pre-manufactured angioplasty balloon that accommodates the patient can be identified and used for treatment.

In some aspects, the present disclosure provides methods for extracting specific geometric measurements from coronary bifurcations. For instance, the methods described in the present disclosure can automate the measurement of important anatomical dimensions in the coronary bifurcation, including two novel measurements: the carina diameter and the tapering length.

In some other aspects, the present disclosure provide methods for generating a model (e.g., a computation model) for predicting specific geometric measurements in diseased anatomy (e.g., vessels narrowed by coronary artery disease) based on data gathered from healthy vessels. For instance, the model can be used to predict values for the carina diameter and tapering lengths for diseased vessels, such as those that have narrowing (e.g., stenosis) at the bifurcation region.

The extracted and/or modeled measurements can be used in a protocol for designing and manufacturing a customized balloon mold. As a non-limiting example, the measurements extracted using the methods described in the present disclosure can be used with the patient-specific medical balloon forming machine and system described in co-pending U.S. Patent Application Publication No. US 2022/0331565, which is herein incorporated by reference in its entirety. For instance, the measurements extracted with the methods descried in the present disclosure can be stored and used as part of a protocol for customizing balloon molds. Additionally, measuring geometries of the tapering regions of bifurcations can provide insight for developing new, more generalized balloon/stent geometries for bifurcations.

The methods described in the present disclosure may also be used in angiographic systems to provide measurements that can be used by operators in clinical decision-making including, but not limited to, decisions regarding interventional strategy, stent sizing, etc. Additionally or alternatively, the measured geometries extracted from medical images using the methods described in the present disclosure can be used for idealized fluid dynamics simulations.

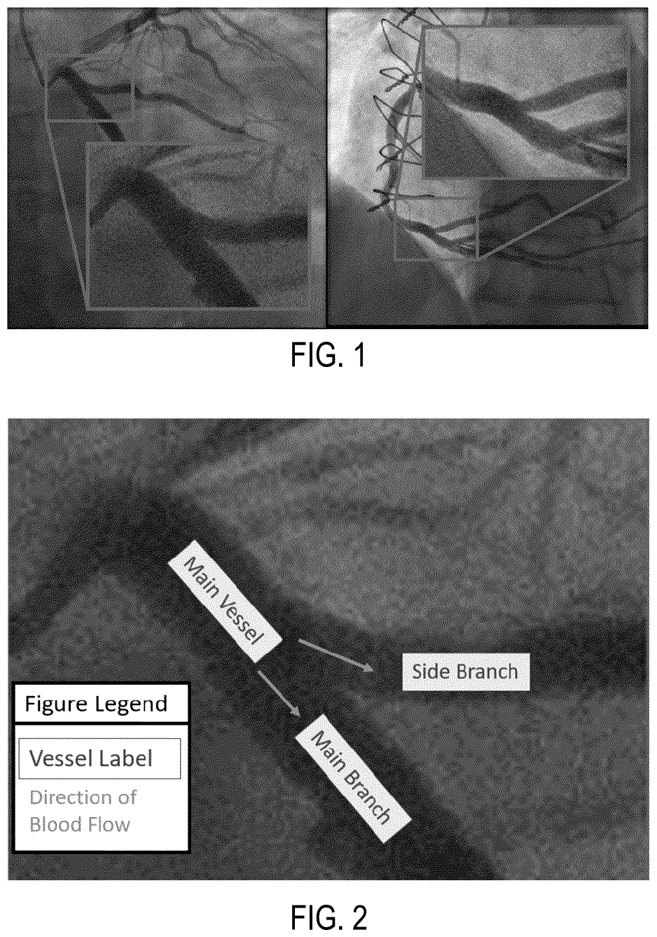

FIG. 1 shows two example medical images that each depict a coronary artery bifurcation. The illustrated medical images are x-ray angiographic images, such as those acquired using an x-ray fluoroscopy imaging system. As other examples, the medical images could be acquired using other angiographic techniques, including magnetic resonance angiography (“MRA”) techniques and computed tomography (“CT”) angiography techniques.

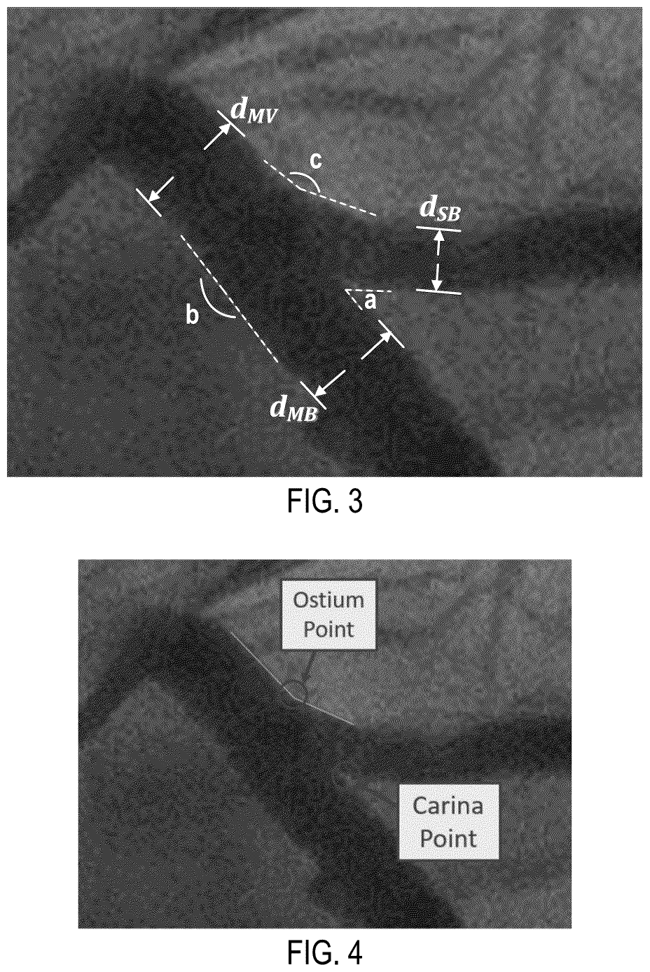

Illustrated in FIG. 2, the most general components of the arterial bifurcation are the main vessel, also the called the parent vessel; the main branch; and the side branch. The main and side branches extend from the main vessel, forming the vessel bifurcation. These terms are illustrated in FIG. 2. Commonly-used parameters for the vessel bifurcation are the diameter of the main vessel d_{MV}, the diameter of the main branch d_{MB}, the diameter of the side branch d_{SB}, and the angles (a, b, c) between the vessels. These diameters and angles are illustrated in FIG. 3.

Additional geometric features that can be used to define the novel measurements are the carina and the ostium of the side branch. The carina of the bifurcation is the point at which the two branches split from the main vessel, which can alternatively be thought of as the tip of the flow divider that splits the parent vessel into two vessels, as shown in FIG. 4. The next parameter, the side branch ostium, is where the side branch splits from the main vessel, also shown in FIG. 4. The carina point and the ostium can be seen most clearly on a two-dimensional (“2D”) sectional view of a vessel bifurcation, where the side branch is defined by two walls. There is the wall of the side branch that intersects with a wall of the main branch, forming the carina, and there is the wall that intersects the wall of the main vessel. Where the side branch wall intersects the main branch wall is considered the ostium of the side branch.

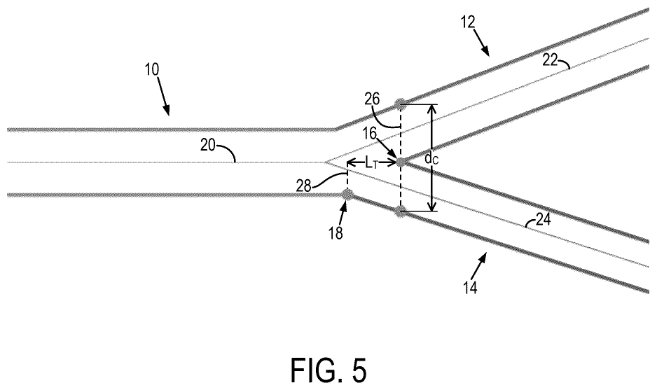

The ostium and the carina definitions aid in describing the novel bifurcation geometry measurements described in the present disclosure, which are called the carina diameter and the tapering distance. FIG. 5 illustrates a diagram depicting a coronary artery bifurcation having main vessel 10, a main branch 12, and a side branch 14. As described above, the carina point 16 is the point where the main branch 12 and the side branch 14 split. The ostium point 18 of the side branch is the point where the side branch 14 splits from the main vessel 10. Shown in FIG. 5 are the main vessel centerline 20, the main branch centerline 22, and the side branch centerline 24.

As shown in FIG. 5, the carina diameter, d_{C}, is defined as the diameter of the vessel at a point passing through the carina point 16 of the vessel, measured along the direction of the main vessel 10. The carina diameter can alternatively be described by defining the centerline of the main vessel (i.e., centerline 20) and defining a carina line 26 in the direction perpendicular to the main vessel centerline 20 and intersecting the carina point 16. Then, the carina diameter is the length of the carina line 26 starting and stopping at the two walls of the vessel (e.g., the outer wall of the main branch 12 and the outer wall of the side branch 14), as shown in FIG. 5.

The tapering length, L_{T}, is defined as the distance from the carina point 16 to the side branch ostium. An ostium line 28 is defined from a point-slope line equation, with the point being the side branch ostium point 18 and the slope being the same slope as the carina line 26. Then, the distance between the carina line 26 and the ostium line 28 may be calculated to determine the length of the vessel taper (i.e., the tapering length, L_{T}).

The carina diameter and the tapering distance have potential use for developing and deploying specifically bifurcation-shaped balloons, where the amount of diameter increase from the main vessel to the bifurcation, and the length of the tapering region could define the anatomical region. The carina diameter represents the maximum expansion diameter a stent placed in the main vessel would need to expand to reach, and the tapering length defines the length along which the stent expands from the main vessel diameter to carina diameter. These two measurements are important for developing customized balloon molds for use in customizable balloon forming machines, such as those described in co-pending U.S. Patent Application Publication No. US 2022/0331565. Also, the carina diameter and tapering length can be clinically advantageous for choosing balloon and stent sizes, whether in conjunction with tapered balloons or with other techniques, such as the final kissing-balloon technique.

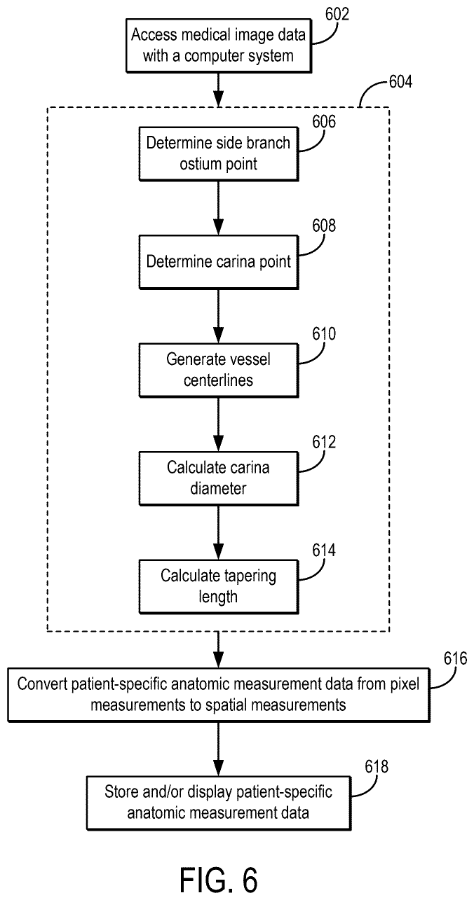

Referring now to FIG. 6, a flowchart is illustrated as setting forth the steps of an example method for estimating geometric measurements of the coronary artery bifurcation from medical images of a patient. With these measurements, a patient-specific balloon design can be generated. As described above, the method can in some instances include two aspects. In a first aspect, bifurcation measurements are extracted from medical images of the patient, including the carina diameter and tapering length measurements described above, which are useful in describing vessel bifurcations. In a second aspect, a simple bifurcation model can be applied to scale geometric parameters in disease anatomy.

The method includes accessing medical image data with a computer system, as indicated at step 602. Accessing the medical image data may include retrieving such data from a memory or other suitable data storage device or medium. Additionally or alternatively, accessing the medical image data may include acquiring such data with a suitable imaging system (e.g., an x-ray fluoroscopy imaging system, a CT system, an MRI system) and transferring or otherwise communicating the data to the computer system, which may be a part of the imaging system.

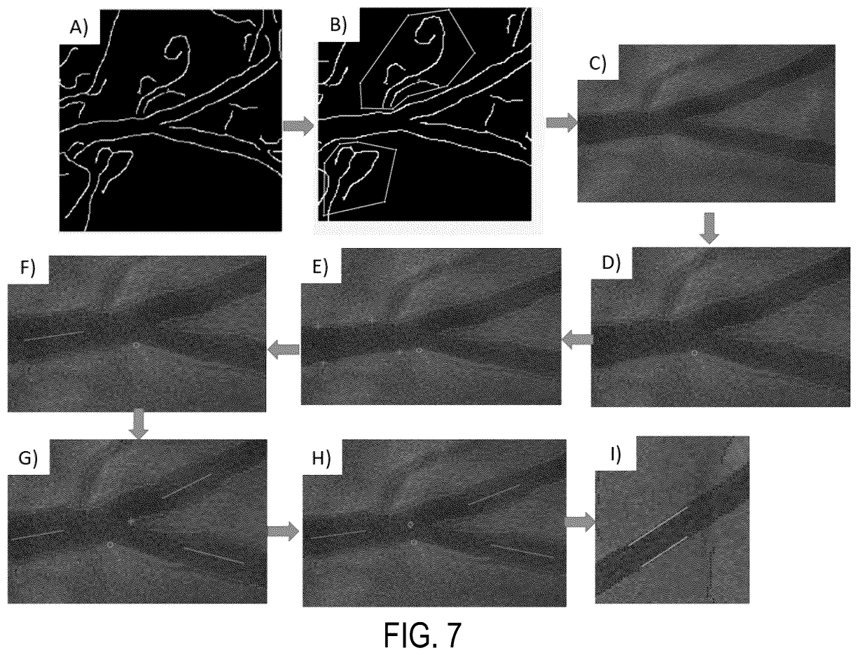

The medical image data are then processed by the computer system to estimate, determine, or otherwise identify various geometric measurements of the coronary artery bifurcation anatomy, as generally indicated by process block 604. FIGS. 7A-7I illustrate various geometric measurements being calculated or otherwise determined from medical image data.

As one example, the side branch ostium point is determined from the medical image data, as indicated at step 606. The side branch ostium point can be determined by generating a user interface with the computer system that prompts a user to review the medical image data and select a point corresponding to the side branch ostium point (FIG. 7D). Alternatively, the side branch ostium point can be automatically selected by processing the medical image data. In some instances, the automatically selected side branch ostium point can then be adjusted by the user via the user interface.

As another example, the carina point is determined from the medical image data, as indicated at step 608. The carina point can be determined by generating a user interface with the computer system that prompts the user to review the medical image data and select a point corresponding to the carina point (FIG. 7H). Alternatively, the carina point can be automatically selected by processing the medical image data. In some instances, the automatically selected carina point can then be adjusted by the user via the user interface.

The vessel centerlines are also estimated or otherwise determined, as indicated at step 610. The centerlines can be manually selected via a user interface, or can be automatically or semi-automatically estimated. As a non-limiting example, the centerlines of the main vessel, the main branch, and the side branch can each be estimated using a digital image processing technique. For instance, the medical image data can first be filtered using an edge detection technique, such as a Canny edge detection technique. The detected edges can then be displayed to the user via the user interface (FIG. 7A). Larger noisy edges around the vessel can be manually or automatically identified and removed using an interactive region-of-interest (“ROI”) tool (FIG. 7B). The edges within the ROI(s) are deleted, and the remaining detected edges can be plotted over the original medical image data (FIG. 7C).

The edges corresponding to the vessel walls are the identified, either manually by the user or in an automatic or semi-automatic fashion. For example, the user can be prompted via the user interface to select four points on the vessel: two points per vessel edge. An example of selected point is shown in FIG. 7E. The edge points between the two user-selected points are then recorded, and straight lines are fitted to the edges. A centerline between the two edges is then calculated. The detected edge points are shown in FIG. 7F as dark blue stars, and the fitted edge and centerlines are shown in light blue. This process can be repeated for each of the main vessel, the main branch, and the side branch, as shown in FIG. 7G

The diameter of the vessel can then be calculated as the distance between the two edge lines. Likewise, the angles (a, b, c) can be calculated by finding the angles between the three centerlines.

Based on the carina point and the vessel centerlines, the carina diameter is calculated or otherwise determined, as indicated at step 612. As described above, to find the carina diameter, a diameter line (i.e., the carina line) is calculated from the slope perpendicular to the main vessel centerline and the carina point. For instance, the carina line is a line perpendicular to the main vessel centerline and intersecting the carina point. Where the carina line intersects the vessel edges is calculated as the carina diameter. The carina diameter line can be displayed to the user via the user interface, as well as the two identified edge points (FIG. 7H). The user is given the opportunity to manually-select the edge points if the carina diameter is incorrectly identified, or to otherwise adjust the measurement as the user deems necessary. The carina diameter is calculated as the distance between the two edge intersection points and recorded.

For vessels that have significant curvature, an additional component can be to use a curved centerline for the main vessel. Then, the carina diameter can be found using the tangent of the centerline near the carina.

Based on the side branch ostium point and the carina line, the taper length is calculated or otherwise determined, as indicated at step 614. An ostium line is calculated using the slope of the carina diameter line and the side branch ostium point. For instance, the ostium line is calculated as a line parallel with the carina diameter line and extending from the side branch ostium point towards the main vessel centerline. The ostium line is displayed to the user via the user interface (FIG. 7H). The distance between the carina diameter line and ostium diameter line is calculated and recorded as the tapering length. For example, since the carina diameter line and the ostium diameter line are parallel, the tapering length can be measured as the distance along a line perpendicular to and extending between the carina diameter line and the ostium diameter line.

In some instances, the measurements made in process block 604 can be converted from pixel-based measurement to absolute measurements using a reference to scale the measurements, as indicated at step 616. For example, when a catheter is visible in the medical image data it can be measured and, because it has a known size, can be used to scale the pixel measurements to absolute measurements, such as millimeters. In such instances, the catheter edges can be found the same way as the vessel edges described above (e.g., with four user-selected points). The edges can then be displayed to the user (FIG. 7J). The user can also be prompted by the user interface to input the French size of the catheter. With the known catheter size, all measurements are converted from pixel to French and/or millimeters.

The resulting patient-specific anatomic measurement data generated by process block 604, and optionally scaled at step 616, are then displayed to a user and/or stored for later use, as indicated at step 618. As an example, all of the described measurements are recorded and displayed to the user. For instance, a table with the angles, vessel diameters, carina diameter, and tapering length can be displayed to the user via the user interface and/or these measurements can be overlaid on the medical image data and displayed to the user via the user interface.

As described above, the patient-specific anatomic measurement data can also be used to generate patient-specific balloon design data, or other patient-specific medical device design data, such as design for patient-specific stents. As one example, a balloon mold can be created using the patient-specific anatomic measurement data to design the dimensions and shape of the mold.

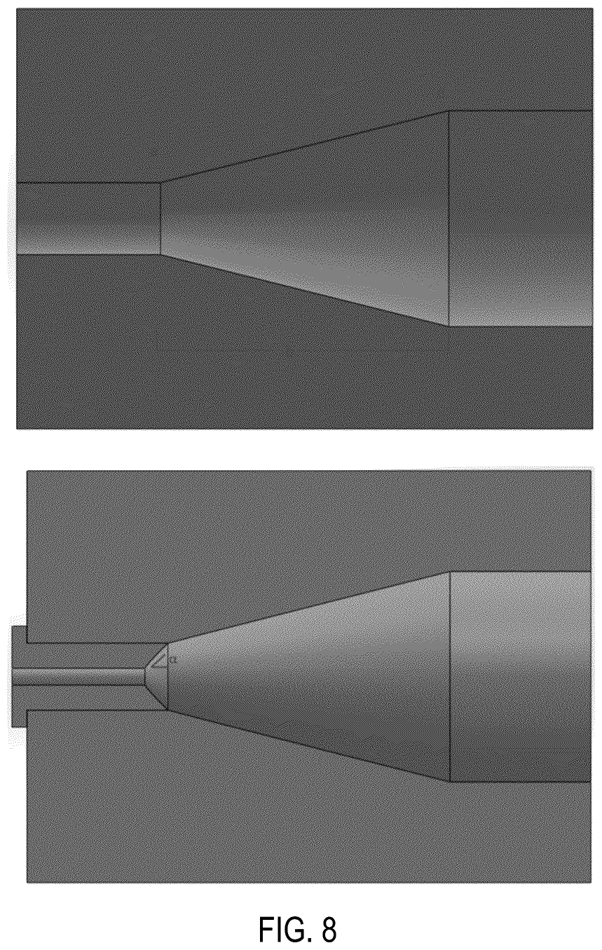

Balloon molds can be created using computer-aided design (“CAD”) tools, as an example. The design of the mold can be a conical shape, with the measurements from the bifurcation and any modeled measurements for diseased anatomy feeding the specific geometry. Shown in FIGS. 8A and 8B, the smaller end of the mold is adjusted to the size of the reference main vessel diameter, the larger end of the mold is adjusted to the size of the measured or modeled carina diameter, and the cone length (i.e., the distance between large and small end) is set as the tapering length. Optionally, the cone length may be some proportion larger than the tapering length to insure adequate balloon length. The mold design may then be created and used as described in co-pending U.S. Patent Application Publication No. US 2022/0331565. FIG. 8 also shows one of two end pieces, which shapes the two ends of the balloon. The angle, α, may be adjusted according to desired balloon shape, but ideally is chosen to minimize stopper length.

As mentioned above, in some implementations, the patient-specific anatomic measurement data can be used to design patient-specific medical devices, such as stent delivery balloons and/or stents. In this way, the patient-specific anatomic measurement data can be used by the computer system to generate patient-specific balloon design data, patient-specific stent design data, or both. Using these, patient-specific balloons, stents, or other medical devices can then be manufactured according to the specifications detailed in the relevant data sets.

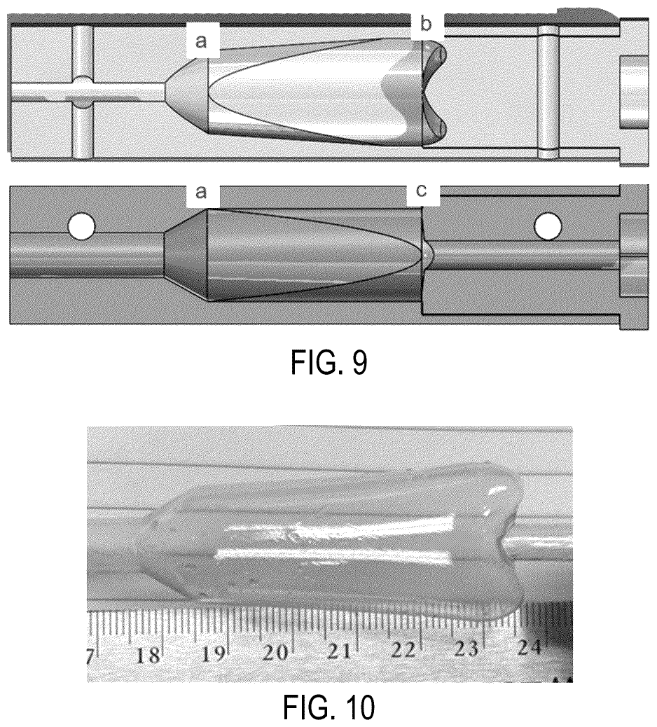

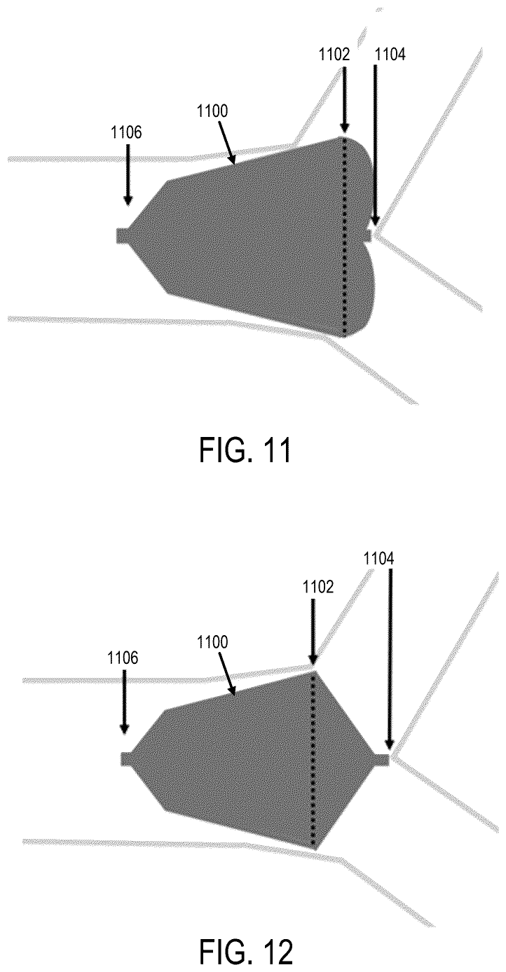

As one example, a stent delivery balloon can be manufactured according to patient-specific balloon data. A stent delivery balloon, also called a “carrier” balloon, is the balloon used to deliver a stent to the bifurcation. An example of a patient-specific carrier balloon that is designed to fit the bifurcation is illustrated in FIG. 9. The balloon tapers from a circular cross section matched to the proximal main vessel diameter (pMVD) to an elliptical shape, with chords defined by the distal main vessel diameter (dMVD) and carina diameter (CD). The balloon uses the pMVD, CD, dMVD, and tapering lengths, which may come from the patient-specific anatomical data generated as described above. The balloon may also feature modified end designs, including two guidewires and a “heart-shape” (e.g., as illustrated in FIGS. 10 and 11) rather than generic tapering (e.g., as illustrated in FIG. 12) which allows the balloon to deliver the stent at or very near to the carina. FIG. 11 illustrates an example of a heart-shaped stent delivery balloon and FIG. 12 illustrates an example of a tapered stent delivery balloon. The distal end 1102 of the balloon body 1100 represents the most distal point the stent can be mounted to. The distal end 1102 may be matched to the carina diameter. The distal guidewire port 1104 is where one or two guidewires may extend into the distal main vessel and side branch. The proximal end 1106 of the balloon body 1100 connects to the guide catheter.

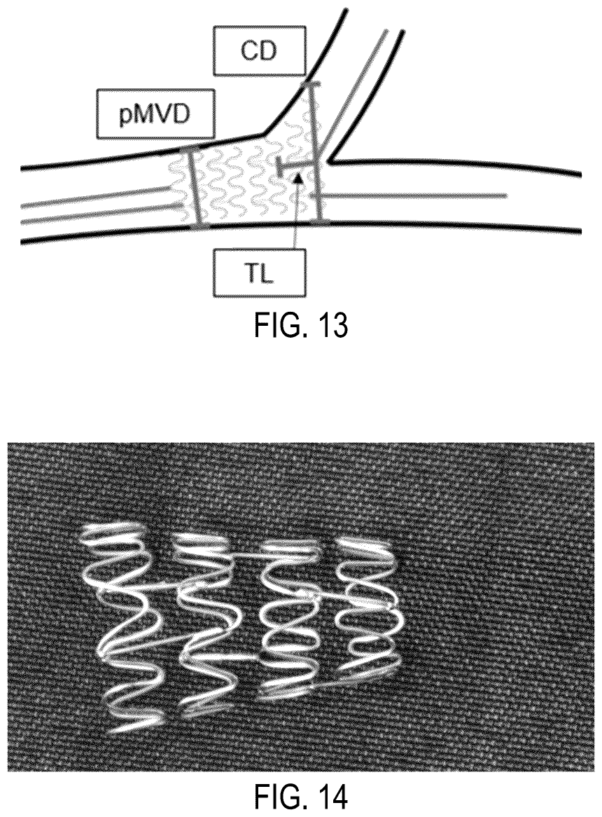

The patient-specific anatomic measurement data, which may include pMVD, CD, and dMVD, may also be used as inputs for custom tapered stent designs. As an example, the stent can be designed to taper from a cylindrical cross section matching the pMVD to an elliptical cross section matching the dMVD and CD, as illustrated in FIG. 13. An example of a tapered stent showing the long axis of the stent's distal end is shown in FIG. 14.

Alternatively, the patient-specific anatomic measurement data may indicate that an off-the-shelf sizing for a balloon and/or stent may be acceptable for the patient. In some instances, for small bifurcation angles the carina diameter may not vary significantly. In these instances, the patient-specific anatomic measurement data may indicate that an “off-the-shelf” option for balloon and stent sizes is acceptable for the patient. The computer system may thus process the patient-specific anatomic measurement data and provide an output indicating the off-the-shelf balloon and/or stent that would best accommodate the patient.

As mentioned above, in some aspects the patient-specific anatomic measurements estimated using the method described with respect to FIG. 6 can be input to a model to generate disease-adjusted patient-specific anatomic measurement data when the patient has narrowing of the coronary arteries, or other disease states that may alter the measurements of certain geometric measurements of the coronary artery bifurcation.

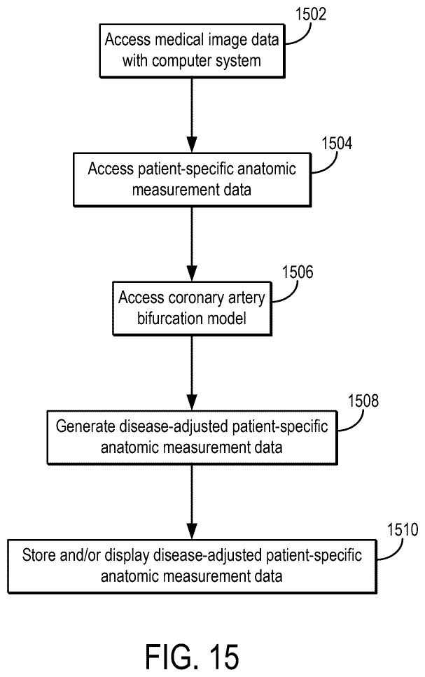

Referring now to FIG. 15, a flowchart is illustrated as setting forth the steps of an example method for generating such disease-adjusted measurement data using a model. The method includes accessing medical image data with a computer system, as indicated at step 1502. Accessing the medical image data may include retrieving such data from a memory or other suitable data storage device or medium. Additionally or alternatively, accessing the medical image data may include acquiring such data with a suitable imaging system (e.g., an x-ray fluoroscopy imaging system, an MRI system) and transferring or otherwise communicating the data to the computer system, which may be a part of the imaging system.

The method also includes accessing previously generated patient-specific anatomic measurement data, as indicated at step 1504. Accessing the patient-specific anatomic measurement data may include retrieving such data from a memory or other suitable data storage device or medium. Additionally or alternatively, accessing the patient-specific anatomic measurement data may include generating such data, as described above, and transferring or otherwise communicating the data to the computer system.

A coronary artery bifurcation model is then accessed with the computer system, as indicated at step 1506. Accessing the coronary artery bifurcation model may include retrieving the model from a memory or other suitable data storage device or medium. Additionally or alternatively, accessing the coronary artery bifurcation model may include generating the model using, in part, the patient-specific anatomic measurement data.

The following is a description of a 2D model of a vessel bifurcation. Inputs to the model are diameters of the vessels (main vessel and the two branches) and the angles between each vessel segment. The model is created visually by placing three straight vessel centerlines using the inputted angles. Next, the vessels are constructed around the centerlines using the given diameters. FIG. 5, for example, shows a visualization of such a model. The model then calculates the carina diameter and the tapering length of the model. These are the “expected” values. The carina diameter and ostium distance may also be calculated individually without constructing the model visualization, though the formulas are derived from the geometric model.

One intended use of the 2D model is to supplement the measurements from the geometric measurements of diseased vessels. The diseased bifurcations requiring intervention tend to have narrowing, especially at the ostial region of the side branch. This disease causes the carina diameter to appear smaller than the original pre-disease carina diameter. Additionally, the location of the side branch ostium can be distorted due to the lesion. The previously described model can be used to predict the original carina diameter and side branch ostium. Inputting the vessel diameters and the angles between the vessels results in a predicted carina diameter and predicted tapering length that can be compared to the measured carina diameters and the measured tapering length.

Thus, disease-adjusted patient-specific anatomic measurement data can then be calculated from the patient-specific anatomic measurement data using the model, as indicated at step 1508. To account for lesions narrowing the vessel at the bifurcation, the angle measurements can be separated from the diameter measurements. The diameters, which may be referred to as “reference diameters” in this embodiment, are measured using the same process as described above, but with the vessel wall points being selected along the vessel, chosen away from any narrowing. This measurement represents the original vessel diameter before disease development in the vessel. Next, the user is prompted to manually place centerlines at the bifurcation core for each of the three vessels. These manually-placed centerlines are used for the angle calculations between vessels. They are placed manually due to vessel curvature, which often makes the centerlines from the reference diameter inaccurate. Additionally, the best-fit centerlines from edge points at the lesion are also often inaccurate, due to lesion eccentricity. The carina diameter and taper lengths are then measured as before, and all measurements (angles, reference diameters, carina diameter, and taper length) are output to the user.

The resulting disease-adjusted patient-specific anatomic measurement data are then displayed to a user and/or stored for later use, as indicated at step 1510. As an example, all of the described measurements are recorded and displayed to the user. For instance, a table with the angles, vessel diameters, carina diameter, and tapering length can be displayed to the user via the user interface and/or these measurements can be overlaid on the medical image data and displayed to the user via the user interface.

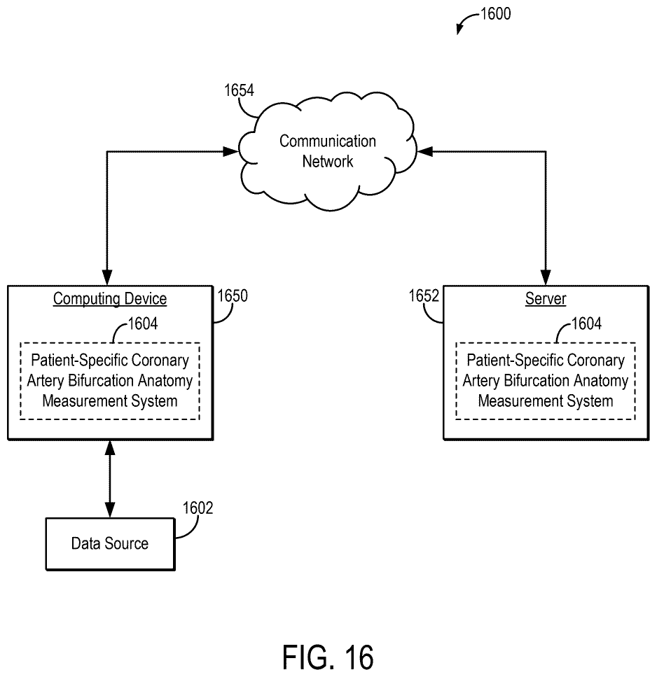

Referring now to FIG. 16, an example of a system 1600 for generating patient-specific measurement of coronary artery bifurcation anatomy, which can be used to design patient-specific angioplasty balloons, in accordance with some embodiments of the systems and methods described in the present disclosure is shown. As shown in FIG. 16, a computing device 1650 can receive one or more types of data (e.g., medical image data) from data source 1602. In some embodiments, computing device 1650 can execute at least a portion of a patient-specific coronary artery bifurcation anatomy measurement system 1604 to generate patient-specific anatomic measurement data indicative of coronary artery bifurcation anatomy from medical image data received from the data source 1602.

Additionally or alternatively, in some embodiments, the computing device 1650 can communicate information about data received from the data source 1602 to a server 1652 over a communication network 1654, which can execute at least a portion of the patient-specific coronary artery bifurcation anatomy measurement system 1604. In such embodiments, the server 1652 can return information to the computing device 1650 (and/or any other suitable computing device) indicative of an output of the patient-specific coronary artery bifurcation anatomy measurement system 1604.

In some embodiments, computing device 1650 and/or server 1652 can be any suitable computing device or combination of devices, such as a desktop computer, a laptop computer, a smartphone, a tablet computer, a wearable computer, a server computer, a virtual machine being executed by a physical computing device, and so on. The computing device 1650 and/or server 1652 can also reconstruct images from the data.

In some embodiments, data source 1602 can be any suitable source of data (e.g., measurement data, images reconstructed from measurement data, processed image data), such as a medical imaging system, another computing device (e.g., a server storing measurement data, images reconstructed from measurement data, processed image data), and so on. In some embodiments, data source 1602 can be local to computing device 1650. For example, data source 1602 can be incorporated with computing device 1650 (e.g., computing device 1650 can be configured as part of a device for measuring, recording, estimating, acquiring, or otherwise collecting or storing data). As another example, data source 1602 can be connected to computing device 1650 by a cable, a direct wireless link, and so on. Additionally or alternatively, in some embodiments, data source 1602 can be located locally and/or remotely from computing device 1650, and can communicate data to computing device 1650 (and/or server 1652) via a communication network (e.g., communication network 1654).

In some embodiments, communication network 1654 can be any suitable communication network or combination of communication networks. For example, communication network 1654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 1654 can be a local area network, a wide area network, a public network (e.g., the Internet), a private or semi-private network (e.g., a corporate or university intranet), any other suitable type of network, or any suitable combination of networks. Communications links shown in FIG. 16 can each be any suitable communications link or combination of communications links, such as wired links, fiber optic links, Wi-Fi links, Bluetooth links, cellular links, and so on.

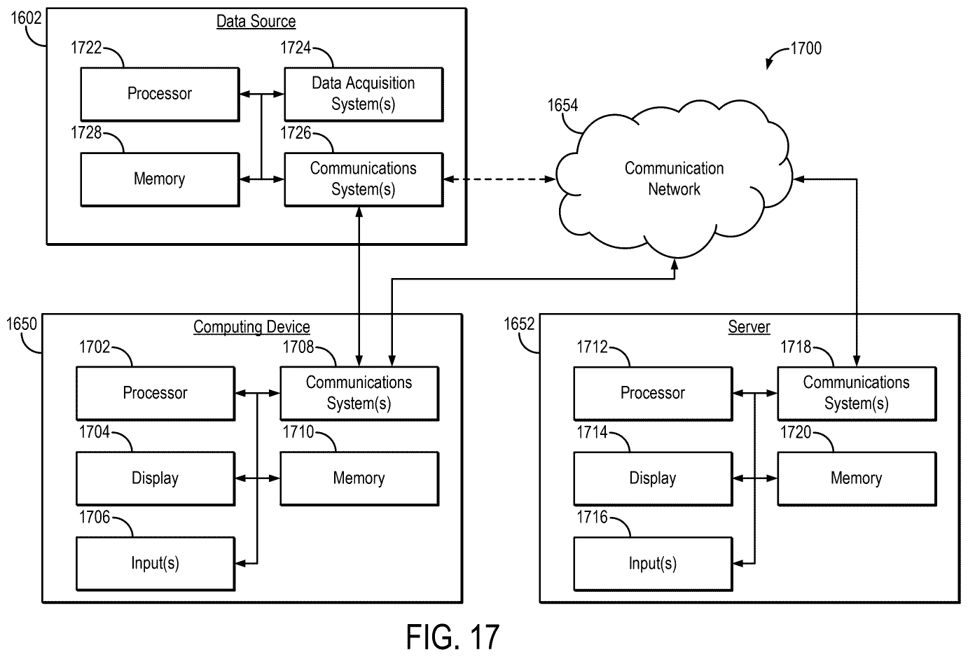

Referring now to FIG. 17, an example of hardware 1700 that can be used to implement data source 1602, computing device 1650, and server 1652 in accordance with some embodiments of the systems and methods described in the present disclosure is shown.

As shown in FIG. 17, in some embodiments, computing device 1650 can include a processor 1702, a display 1704, one or more inputs 1706, one or more communication systems 1708, and/or memory 1710. In some embodiments, processor 1702 can be any suitable hardware processor or combination of processors, such as a central processing unit (“CPU”), a graphics processing unit (“GPU”), and so on. In some embodiments, display 1704 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 1706 can include any suitable input devices and/or sensors that can be used to receive user input, such as a keyboard, a mouse, a touchscreen, a microphone, and so on.

In some embodiments, communications systems 1708 can include any suitable hardware, firmware, and/or software for communicating information over communication network 1654 and/or any other suitable communication networks. For example, communications systems 1708 can include one or more transceivers, one or more communication chips and/or chip sets, and so on. In a more particular example, communications systems 1708 can include hardware, firmware, and/or software that can be used to establish a Wi-Fi connection, a Bluetooth connection, a cellular connection, an Ethernet connection, and so on.

In some embodiments, memory 1710 can include any suitable storage device or devices that can be used to store instructions, values, data, or the like, that can be used, for example, by processor 1702 to present content using display 1704, to communicate with server 1652 via communications system(s) 1708, and so on. Memory 1710 can include any suitable volatile memory, non-volatile memory, storage, or any suitable combination thereof. For example, memory 1710 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 1710 can have encoded thereon, or otherwise stored therein, a computer program for controlling operation of computing device 1650. In such embodiments, processor 1702 can execute at least a portion of the computer program to present content (e.g., images, user interfaces, graphics, tables), receive content from server 1652, transmit information to server 1652, and so on. For example, the processor 1702 and the memory 1710 can be configured to perform the methods described herein (e.g., the method of FIG. 6, the method of FIG. 15).

In some embodiments, server 1652 can include a processor 1712, a display 1714, one or more inputs 1716, one or more communications systems 1718, and/or memory 1720. In some embodiments, processor 1712 can be any suitable hardware processor or combination of processors, such as a CPU, a GPU, and so on. In some embodiments, display 1714 can include any suitable display devices, such as an LCD screen, LED display, OLED display, electrophoretic display, a computer monitor, a touchscreen, a television, and so on. In some embodiments, inputs 1716 can include any suitable input devices and/or sensors that can be used to receive user input, such as a keyboard, a mouse, a touchscreen, a microphone, and so on.

In some embodiments, communications systems 1718 can include any suitable hardware, firmware, and/or software for communicating information over communication network 1654 and/or any other suitable communication networks. For example, communications systems 1718 can include one or more transceivers, one or more communication chips and/or chip sets, and so on. In a more particular example, communications systems 1718 can include hardware, firmware, and/or software that can be used to establish a Wi-Fi connection, a Bluetooth connection, a cellular connection, an Ethernet connection, and so on.

In some embodiments, memory 1720 can include any suitable storage device or devices that can be used to store instructions, values, data, or the like, that can be used, for example, by processor 1712 to present content using display 1714, to communicate with one or more computing devices 1650, and so on. Memory 1720 can include any suitable volatile memory, non-volatile memory, storage, or any suitable combination thereof. For example, memory 1720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1720 can have encoded thereon a server program for controlling operation of server 1652. In such embodiments, processor 1712 can execute at least a portion of the server program to transmit information and/or content (e.g., data, images, a user interface) to one or more computing devices 1650, receive information and/or content from one or more computing devices 1650, receive instructions from one or more devices (e.g., a personal computer, a laptop computer, a tablet computer, a smartphone), and so on.

In some embodiments, the server 1652 is configured to perform the methods described in the present disclosure. For example, the processor 1712 and memory 1720 can be configured to perform the methods described herein (e.g., the method of FIG. 6, the method of FIG. 15).

In some embodiments, data source 1602 can include a processor 1722, one or more data acquisition systems 1724, one or more communications systems 1726, and/or memory 1728. In some embodiments, processor 1722 can be any suitable hardware processor or combination of processors, such as a CPU, a GPU, and so on. In some embodiments, the one or more data acquisition systems 1724 are generally configured to acquire data, images, or both, and can include a medical imaging system (e.g., an x-ray fluoroscopy system, an MRI system). Additionally or alternatively, in some embodiments, the one or more data acquisition systems 1724 can include any suitable hardware, firmware, and/or software for coupling to and/or controlling operations of a medical imaging system. In some embodiments, one or more portions of the data acquisition system(s) 1724 can be removable and/or replaceable.

Note that, although not shown, data source 1602 can include any suitable inputs and/or outputs. For example, data source 1602 can include input devices and/or sensors that can be used to receive user input, such as a keyboard, a mouse, a touchscreen, a microphone, a trackpad, a trackball, and so on. As another example, data source 1602 can include any suitable display devices, such as an LCD screen, an LED display, an OLED display, an electrophoretic display, a computer monitor, a touchscreen, a television, etc., one or more speakers, and so on.

In some embodiments, communications systems 1726 can include any suitable hardware, firmware, and/or software for communicating information to computing device 1650 (and, in some embodiments, over communication network 1654 and/or any other suitable communication networks). For example, communications systems 1726 can include one or more transceivers, one or more communication chips and/or chip sets, and so on. In a more particular example, communications systems 1726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1728 can include any suitable storage device or devices that can be used to store instructions, values, data, or the like, that can be used, for example, by processor 1722 to control the one or more data acquisition systems 1724, and/or receive data from the one or more data acquisition systems 1724; to generate images from data; present content (e.g., data, images, a user interface) using a display; communicate with one or more computing devices 1650; and so on. Memory 1728 can include any suitable volatile memory, non-volatile memory, storage, or any suitable combination thereof. For example, memory 1728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1728 can have encoded thereon, or otherwise stored therein, a program for controlling operation of data source 1602. In such embodiments, processor 1722 can execute at least a portion of the program to generate images, transmit information and/or content (e.g., data, images, a user interface) to one or more computing devices 1650, receive information and/or content from one or more computing devices 1650, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for generating anatomic measurements of a coronary artery bifurcation from medical image data, comprising:
(a) accessing medical image data with a computer system, wherein the medical image data depict a coronary artery bifurcation in a patient;
(b) processing the medical image data with the computer system to estimate a centerline of a main vessel of the coronary artery bifurcation;
(c) determining in the medical image data, by the computer system, a carina point indicating a split between a main branch and a side branch of the coronary artery bifurcation;
(d) calculating a carina diameter in the medical image data using the main vessel center line and the carina point;
(e) determining in the medical image data, by the computer system, a side branch ostium point indicating a split between the main vessel and the side branch of the coronary artery bifurcation;
(f) calculating a tapering length in the medical image data using the carina diameter and the side branch ostium point;
(g) storing the carina diameter and the tapering length as patient-specific anatomic measurement data of the coronary artery bifurcation.

2. The method of claim 1, further comprising generating a user interface with the computer system and presenting the patient-specific anatomic measurement of the coronary artery bifurcation to a user via the user interface.

3. The method of claim 1, further comprising converting the patient-specific anatomic measurement data to patient-specific angioplasty balloon design data using the computer system.

4. The method of claim 3, further comprising creating an angioplasty balloon mold using the patient-specific angioplasty balloon design data to dimension and shape the angioplasty balloon mold.

5. The method of claim 3, wherein converting the patient-specific anatomic measurement data to the patient-specific angioplasty balloon design data using the computer system comprises scaling the patient-specific anatomic measurement data from pixel measurements to spatial measurements.

6. The method of claim 5, wherein converting the patient-specific anatomic measurement data to the patient-specific angioplasty balloon design data comprises identifying a catheter depicted in the medical image data, prompting a user for measurements of the catheter via the computer system; and scaling the patient-specific anatomic measurement data based on the measurements input by the user.

7. The method of claim 1, wherein calculating the carina diameter comprises:
determining a line segment that intersects the carina point, is perpendicular to the main vessel centerline, and spans from a first vessel wall edge on a first side of the main vessel centerline to a second vessel wall edge opposite the main vessel centerline in the medical image data; and
calculating a length of the line segment in the medical image data as the carina diameter.

8. The method of claim 7, wherein calculating the tapering length comprises:
determining a second line segment that intersects the side branch ostium point and is parallel to the line segment intersecting the carina point in the medical image data; and
calculating a distance in the medical image data between the line segment intersecting the carina point and the second line segment as the tapering length.

9. The method of claim 1, further comprising adjusting the patient-specific anatomic measurement data of the coronary artery bifurcation for disease-related anatomical changes using a model of the coronary artery bifurcation.

10. The method of claim 9, wherein adjusting the patient-specific anatomic measurement data of the coronary artery bifurcation using the model comprises:
calculating vessel diameters in the medical image data comprising a main vessel diameter, a main branch diameter, and a side branch diameter;
calculating vessel angles in the medical image data comprising an angle between the main vessel and the main branch, an angle between the main vessel and the side branch, and an angle between the main branch and the side branch;
inputting the vessel diameters and vessel angles to the model using the computer system, generating predicted carina diameter and predicted tapering length values as an output;
comparing the carina diameter to the predicted carina diameter and the tapering length to the predicted tapering length; and
adjusting the patient-specific anatomic measurement data of the coronary artery bifurcation based on the comparison.

11. A method for designing a mold for use in manufacturing an angioplasty balloon, comprising:
(a) accessing medical image data of a patient using a computer system, wherein the medical image data depict a coronary artery bifurcation in a patient;
(b) processing the medical image data with the computer system to
calculate a carina diameter in the medical image data by the computer system, wherein the carina diameter spans the coronary artery bifurcation from a first vessel wall to an opposed vessel wall passing through a carina point of the coronary artery bifurcation and perpendicular to a centerline of a main vessel of the coronary artery bifurcation;
calculate a tapering length in the medical image data by the computer system, wherein the tapering length is a distance between the carina diameter line and a parallel line in the medical image data intersecting a side branch ostium point;
(c) generating balloon mold design data using the computer system, wherein the balloon model design data comprise instructions to manufacture a balloon mold including a conical portion having a large end, a small end, and a tapered portion extending between the large and the small end;
(d) adjusting the balloon mold design data with the computer system by sizing the large end of the conical portion according to the carina diameter and sizing a distance between the large end and the small end of the conical portion based on the tapering length.

12. The method of claim 11, further comprising manufacturing a balloon mold according to the balloon mold design data.

13. The method of claim 12, further comprising manufacturing a patient-specific angioplasty balloon using the balloon mold.
